(19) United States
(12) Patent Application Publication
(10) Pub. No.: US 2007/0062538 A1
(43) Pub. Date: Mar. 22, 2007

(54) Undetectable nasal insert

(76) Inventors: Dave Foggia, La Grande, OR (US); Michael C. Stewart, Santa Fe, NM (US)

Correspondence Address: Dave Foggia, 1002 3rd Street, La Grande, OR, 97850, US

(21) Appl. No.: 11/524,765

(22) Filed: Sep. 20, 2006

Related U.S. Application Data
(60) Provisional application No. 60/719,494, filed on Sep. 21, 2005.

(51) Int. Cl. A61M 15/08

(52) U.S. Cl. 128/207.18

(57) ABSTRACT

An intranasal insert of the type having two insert bodies (10) which are then connected together by a transparent non-rigid elongated fiber (24). The transparent fiber provides for comfort to the user and allows the insert to be worn without being visible to others. The insert bodies generally contain a substance (32) for drug delivery or control of foul odors and are placed inside the nose, one through each nostril (26). The design of the insert allows for easy removal from the nose and sanitary disposal by blowing into a clean tissue after use.

### CROSS-REFERENCE TO RELATED APPLICATIONS

This application claims the benefit of provisional patent application Ser. No. 60/719,494, filed Sep. 21, 2005 by the present inventors.

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates to nasal devices, specifically to intranasal inserts.

2. Prior Art

People have been trying to control foul odors in various ways for ages. Drug companies are now looking to the human nose more and more for drug delivery. Conventional dust masks irritate the face and they invariably leak to some degree. Swimmers nose plugs are uncomfortable to wear and often unsightly. Effective snoring devices use adhesives which are uncomfortable to use. Inventors of the past have tried to address some of these issues with various inventions. However, the nose and people for that matter are very sensitive. The fact is, intranasal devices of the past have simply not accommodated for comfort or discreetness.

U.S. Pat. No. 6,015,425 shows an air freshener insert for use during dental procedures that is referred to as a intranasal clip. The clip is comprised of a “bendable arcuate band” which is supposedly closed around the user's nasal septum. The band further has “a pair of reverse curvatures” which are used to hold a pair of “odorant pads” in a fixed position away from the septum wall. This invention is essentially a rigid form that firmly holds two pads in a given position and is inserted into or clamped onto the nose. In other words, it is similar to many other rigid forms in that it holds a particular shape prior to insertion into the nose. There are several important problems with this concept. The first being that the nose is extremely sensitive, and rigid forms are simply always felt by the user. The band of this device would be no exception to this fact. Second, this device would have limited adjustability of its “pads” because of the rigid nature of the band which holds them in position. If the device were inserted into the nose and it did not feel right, it would need to be removed from the nose by pulling on the band with the fingers. It would then be adjusted and re-inserted back into the nose. This fact is a nuisance and a clear sanitary problem. Lastly, the band of this device would be easily detected by anyone looking at the user. It would not have the superior advantage of being out of view and therefore discreet.

U.S. Pat. No. 1,311,461 and U.S. Pat. No. 3,463,149 both show nasal inserts which primarily function as nasal filters but can also be used as inhalers. Respectively, the first uses “disposed pads or sponges adapted to fit the nostrils, and yoke having its ends formed with spirals adapted to screw into said pads and retain them in proper position”. The second is claimed as being singular, and utilizes “a nose air filter, the combination of a mass of cotton filler enclosed within a porous covering to form a porous case of elongated character and having an apex at an upper end”. Both devices utilize large pads which fill the nasal vestibule area of the nose and act to filter particles from the incoming air. Both devices also utilize bendable wire forms which hold their pads so as “to retain them in proper position.” Again, this use of rigid forms would simply be too uncomfortable for most users. Wearing such a device produces the sensation that something is hanging off the end of the nose. Additionally, such forms are irritating to the septum walls when they make contact. These irritations would not be acceptable to most people. Further, these devices would again exhibit the major disadvantage of being highly visible while being worn.

U.S. Pat. No. 406,580, U.S. Pat. No. 831,004, U.S. Pat. No. 1,579,486, U.S. Pat. No. 1,709,740, and U.S. Pat. 5,922,006 are all examples of nasal inserts that act as nasal inhalers or nasal dilators. All use two insertable sections that are connected by a bendable wire. The bendable wire again holds a particular shape prior to insertion into the nose. This has the same major disadvantages as stated above. Heretofore, all known nasal inserts suffer from generally three main disadvantages:

(a) Through use of rigid forms they are uncomfortable to wear. The interior walls of the nose along with the hairs which line them are extremely sensitive. Further, the nose is in a constant state of movement through inhaling and exhaling of air. When rigid forms are used, they are constantly felt by the user because they are unable to “move” with the nose. Therefore, they become obstructions to the nose and its operation.

(b) They incorporate rigid forms that are difficult to adjust while inserted into the nose. Once the insert is inside the nose, there is really very little ability to adjust the individual pads. The only way to adjust the insert is to push or pull on the rigid form that is connected to each pad. As this will generally not solve the problem, the insert must be removed, handled with fingers, and “bent”. Then it is re-inserted into another uncomfortable position inside the nose.

(c) They incorporate a connecting means between each of the insertable sections that is highly visible. The nose is a rather “private” area of the body. Most people prefer not to draw attention to it. Nasal inserts of the past have all been highly visible which will never be accepted in the main stream of society.

### BACKGROUND OF THE INVENTION 3. Objects and Advantages

Accordingly, similar to the objects and advantages of the disposable nasal insert described in our above patent, several objects and advantages of the present invention are:

(a) To provide a nasal insert that is comfortable to wear;

(b) To provide a nasal insert where the inserted pads can be individually adjusted inside the nose, without having to remove the insert to adjust;

(c) To provide a nasal insert that moves with the nose and offers relatively no resistance to it.

(d) To provide a nasal insert that when installed into position is difficult for most people to see or detect;

(e) To provide a nasal insert that is easily removed from the nose; and

(f) To provide a nasal insert that is difficult to see and will break down quickly if discarded into the environment.

Further objects and advantages are to provide a nasal insert that is extremely easy and convenient to use for large everyday applications such as drug deliver, personal protection against odors, and air filtering for particulates such as pollen. To provide a self-administered nasal insert that is inexpensive to manufacture, sterile, and individually packaged where it can be placed in dispensers to benefit workers of industry from factory workers to hospital staff. Further, to provide the everyday person with a convenient breathing aid or drug delivery form that can be easily placed in a purse, shirt pocket, tool box, glove compartment, medicine chest, etc. until needed. Still further objects and advantages will become apparent from a consideration of the ensuing description and drawings.

### SUMMARY

In accordance with the present invention an intranasal insert comprises two insert bodies which are placed in the human nose, one through each nostril, and a non-rigid elongated fiber connects the two insert bodies together.

### DRAWINGS—FIGURES

In the drawings, closely related figures have the same number but different alphabetical suffixes.

FIGS. 1A and 1B show perspective views of a preferred embodiment of the invention.

FIGS. 2A to 2E are cross-sectional views of a human nose showing variations of the preferred embodiment installed.

FIGS. 3A to 3F are perspective views of further variations of the preferred embodiment in uninstalled position.

FIGS. 4A and 4B are left side and bottom perspective views of a human nose respectively with the preferred embodiment installed.

FIGS. 5A to 5J are perspective views of alternative embodiments in uninstalled position.

### DRAWINGS—REFERENCE NUMERALS

| | | | |
|---|---|---|---|
| 10 | insert body | 12 | end regions |
| 14 | nose cross-section | 16 | knot |
| 18 | restraining clip | 20 | adhesive |
| 22 | septum | 24 | fiber |
| 26 | nostrils | 28 | core |
| 30 | electronics | 32 | substance |
| 34 | pull member | 36 | subbody |

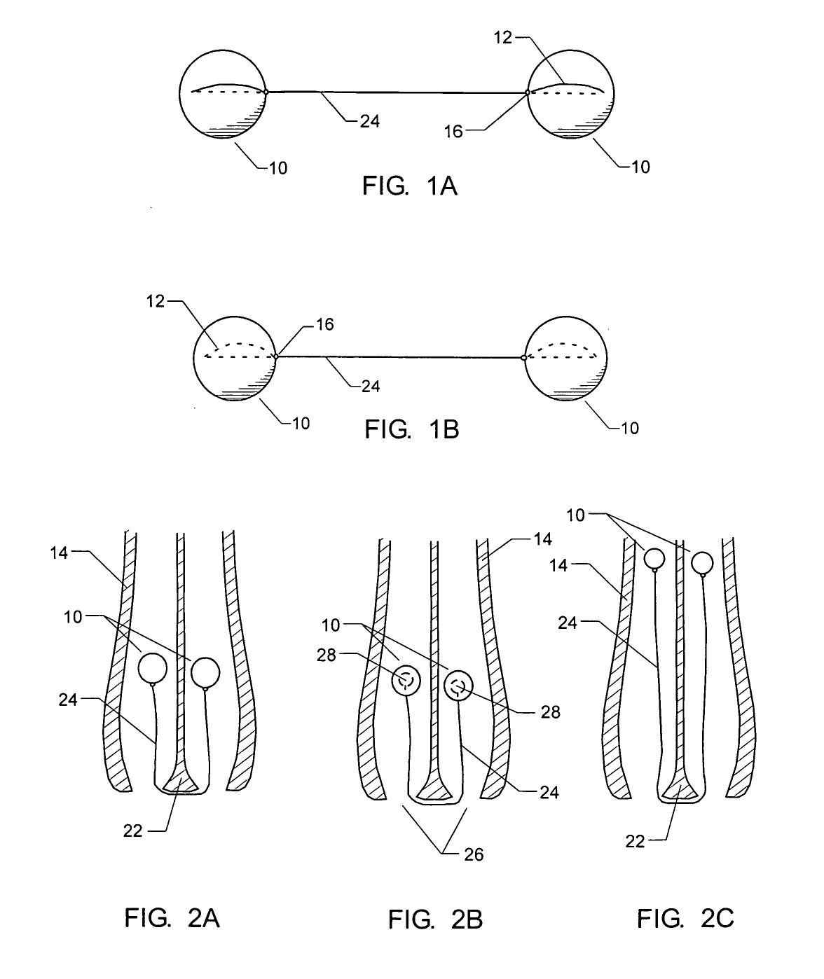

### DETAILED DESCRIPTION—FIGS. 1A AND 1B—PREFERRED EMBODIMENT

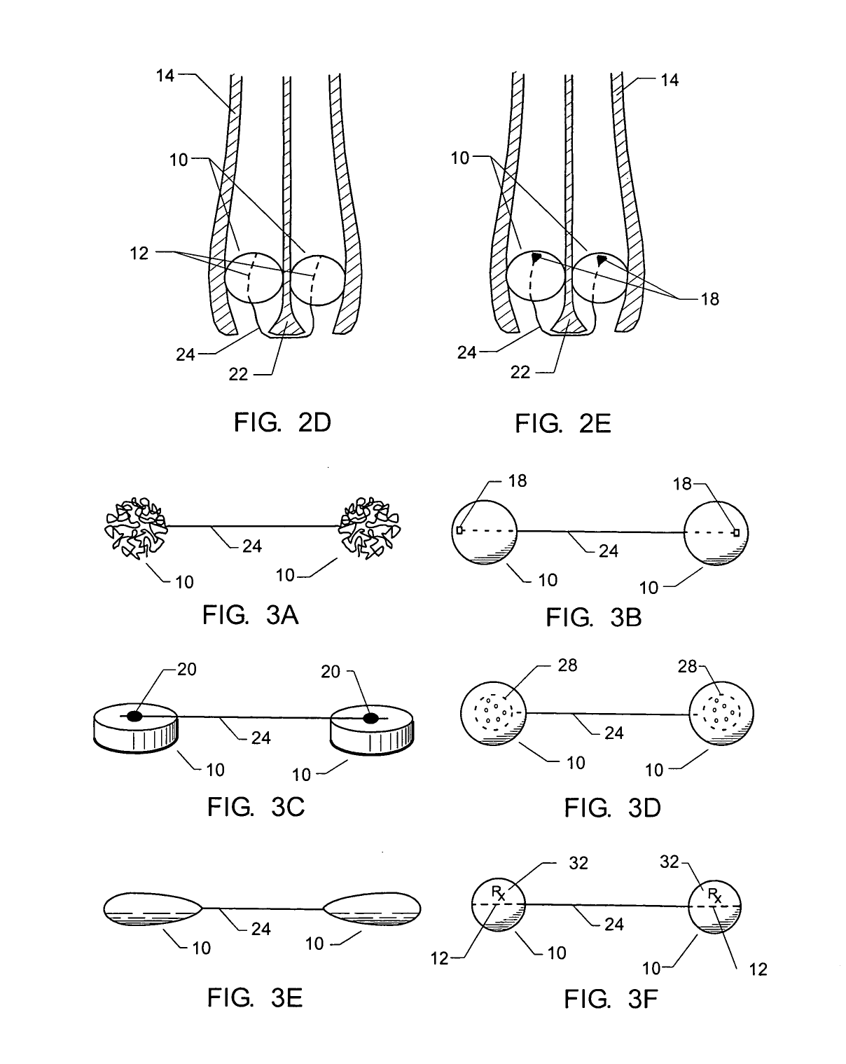

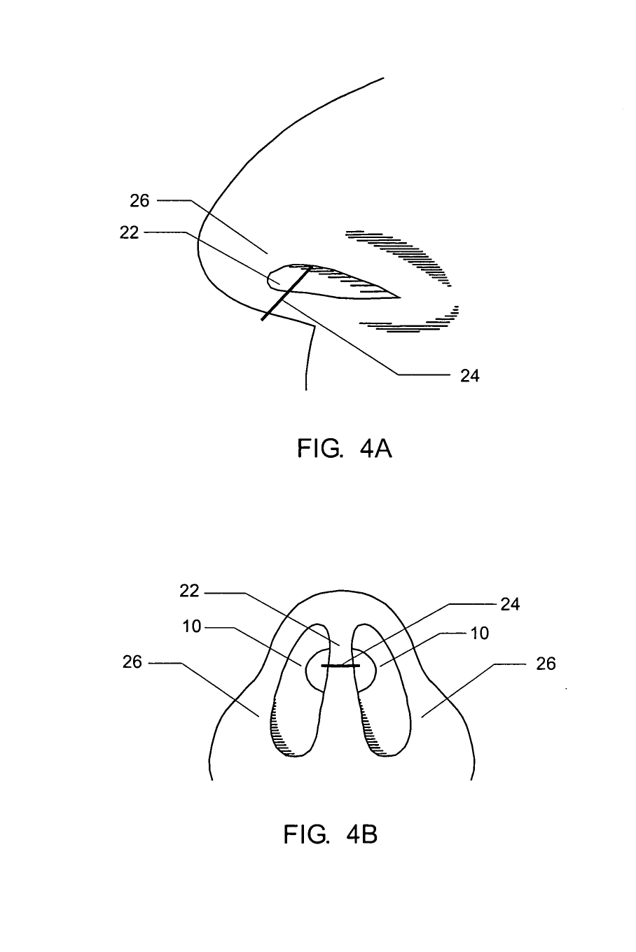

A preferred embodiment of the intranasal insert of the present invention is illustrated in FIG. 1A (top perspective view), FIG. 1B (side perspective view ). The insert has generally two spherical insert bodies 10 which are usually made of soft materials such as cotton or a medical grade foam and can widely vary in size and shape depending upon the purpose of the insert bodies. When the purpose of the insert bodies is to deliver drugs or scents into the respiratory system, each insert body 10 is generally sized about 7mm in diameter for adults (FIGS. 2A, 2B). When the purpose of the insert bodies is to filter inhaled air (FIG. 2D) or to block airflow through the nose altogether (FIG. 2E), each of the bodies has elastic qualities and can be approximately 20 mm in diameter. In the preferred embodiment the insert bodies 10 are generally soft cotton “poms”. However, they can also be made of different materials from soft medical grade foams (FIG. 3E) to bodies molded out of medications that may dissolve inside the nasal passages (FIG. 3F), either of which may contain a core 42 (FIG. 2B). The bodies can also be color coded and marked for identification purposes similar to the way different pharmaceutical companies color and mark pills, etc. (FIG. 3F).

Connecting insert bodies 10 together is generally one predetermined length of clear transparent fiber 24. The fiber is very thin monofilament nylon line of an approximate diameter of 0.005 inches or 0.13 mm such as that commonly used in textile quilting. Fiber 24 can be made out of any material that exhibits the quality of being “non-rigid”. That is to say that when the insert is picked up with the thumb and forefinger of the human hand, fiber 24 will visually bend. The length of fiber 24 is generally 1 to 2.5 inches in length for adults and forms two ends which can be connected in numerous ways to the insert bodies 10 along end regions 12. In the preferred embodiment, the fiber is sewn through the insert bodies. An example of this is given where the fiber is then tied back upon itself at knot 16. This method of securing the insert bodies to the fiber is one method among many. Other methods would include for example, molding foam or other materials directly onto the fiber, using an adhesive between the insert bodies and the fiber, molding the insert bodies and fiber out of the same material, producing them from a single length of material thus being continuous, etc.

### Operation—FIGS. 2A-2E

The manner of installing the intranasal insert of the present invention is simple. First of all, the user grasps the insert by holding each insert body 10 independently by using the thumb and forefinger of each hand. Then, the user lifts his/her hands carrying each of the insert bodies up just outside and under the nostrils of their nose. The user then pushes each of the insert bodies in and up against the ceiling of the nose, one through each nostril 26, using the thumb from each hand. This is then generally followed by using the smallest finger of each hand for further placement. If additional adjustment is necessary, the user can further re-position either insert body while inside the nose by pressing with his/her fingers on the outside of the nose. Each insert body can be felt under the skin of the nose and can be moved somewhat by pressing with the fingers. Lastly, additional seating of the insert bodies can occur if the user pulls slightly on the connecting means or inhales airflow sharply as is normally and regularly done with the nose. Sharply inhaling airflow has the tendency to drive the bodies inside the nose and is also done in the case where a smaller size insert body 10 is used in combination with a longer fiber 24 for purposes such as drug delivery deeper inside the nasal passages (FIG. 2C). If the insert is used for filtering inhaled air or to block airflow altogether, entering or leaving the nose, each insert body 10 is made larger to fill a section of airway with a suitable material such as elastic foam (FIGS. 2D, 2E).

In either of these cases, the user must first compress these larger insert bodies by squeezing them with the fingers prior to insertion into the nose.

During use, the insert in the preferred embodiment can be worn without anyone around the user visually identifying its presence. This is mainly due to the transparent nature of the material used for fiber 24. It is also due to the non-rigid characteristics of the fiber which enable it to adjust neatly and comfortably around the exterior portion of septum 22. Consequently, if the user feels the need to scratch or rub their nose during use, this can safely be done and the insert will adjust and generally remain in place. Fiber 24 will not have a tendency to break. Further, because of the safety and comfort characteristics of the fiber, the user can confidently leave the insert in the nose overnight. This is an important advantage for different uses including snoring prevention and drug delivery.

To remove the insert from the nose, one can simply use a clean tissue and firmly “blow” the nose in the identical manner that is normally done with tissue. Using this technique is preferred because it is sanitary and it allows for discreet disposal of the used insert while in the presence of others. If one does not have a clean tissue or other material, the alternative is to grasp fiber 24 with fingers and pull the insert away from the nose for disposal.

### FIGS. 3A-3F—Additional Embodiments

Additional embodiments are shown as perspective views in FIGS. 3A, 3B, 3C, 3D, 3E, 3F; In FIG. 3A an example is given of an insert where each insert body 10 and fiber 24 are made from a continuous length of material, such as sewing thread; In FIG. 3B an insert is shown of spherically shaped foam insert bodies with fiber 24 passing through their interiors where a restraining clip 18 has been added onto the fiber in the manufacturing process to prevent the insert bodies from sliding off the ends of the fiber; In FIG. 3C an insert is shown where an adhesive 20 is used between wafer shaped insert bodies and the fiber; In FIG. 3D an insert is shown having a plastic perforated core 28 which is connected directly with the fiber and where the core is encased in a soft foam; FIG. 3E shows an example of an insert where the insert bodies are tear drop shaped; FIG. 3F shows one example of an insert where the insert bodies are made up of a substance 32, such as a medication, that may be molded onto the ends of the connecting means and that can eventually dissolve or be released inside the nose.

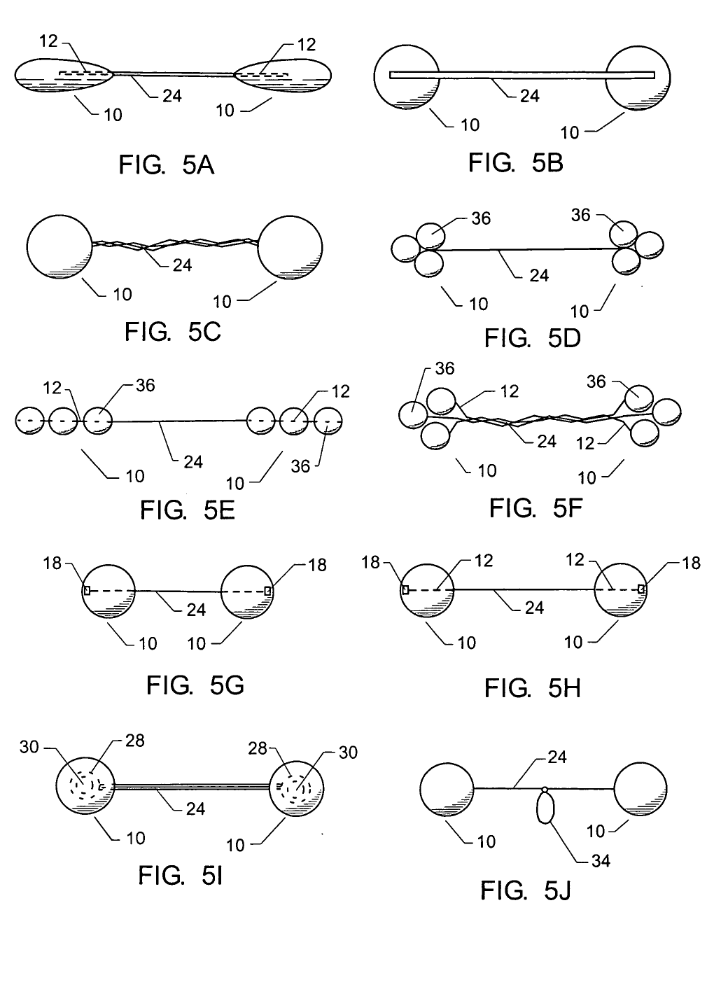

### FIGS. 5A-5J—Alternative Embodiments

There are several different ways to vary the insert of the present invention to achieve similar results as discussed above. By using other transparent materials for the connecting means is one method. FIG. 5A shows an example of using very thin clear transparent rubber tubing for fiber 24; FIG. 5B shows an insert where fiber 24 is made of transparent film which is bonded to the insert bodies.

The insert can be varied by increasing the number of lengths of fiber that make up the connecting means. FIG. 5C shows a simple example of an insert where the connecting means is braided.

The insert can be modified so that each of the insert bodies on either end of the connecting means can be made up of more than one subbody 36. FIG. SD shows an insert with three subbody 36 elements connected together at the end of the connecting means to form each of the insert bodies 10; FIG. SE shows an insert with three subbody 36 elements not connected together at the end of the connecting means; FIG. 5F shows an insert with three subbody 36 elements not connected together at each end with the connecting means made up of three lengths of material braided together to form fiber 24.

The connecting means can also be made to be adjustable as mentioned in our above patent. FIG. 5G shows an insert where fiber 24 is in an un-stretched position made of a material that will stretch and is non-elastic. FIG. 5H shows the same insert and fiber 24 in a stretched out position, thus giving one example of an extendable connecting means.

Other alternatives include FIG. 5I showing an insert wherein each of the insert bodies comprises a foam encased core 28 filled with electronics 30 whereby each of the insert bodies is then connected together using a plurality of lengths of optical fiber forming fiber 24. FIG. 5J shows an example where a section of fiber 24 has been made into a loop to form pull member 34 to provide a grasping object for the fingers to remove the insert from the nose.

Advantages

From the description above, a number of advantages of our intranasal insert become evident:

(a) It is more comfortable to install since there is no rigid connecting means that can make contact and irritate the user's nasal septum.

(b) It is more comfortable to wear since the connecting means is usually very thin and flexible and draws neatly in front of the exterior nasal septum. The user does not get the unnerving sense that something is “hanging” off the end of the nose as is the case with other inserts.

(c) It is far easier to adjust than other inserts because it allows the user the ability to adjust the individual positions of the insert bodies without removing the insert from the nose. This is done by pressing on the outside of the nose with one's fingers to move the insert bodies.

(d) It is easier to remove from the nose because there is no rigid connecting means that is “clamped” around the septum or that can be caught or hung up in the nose upon removal.

(e) When the connecting means is made of transparent material, it allows the user the superior ability to wear the insert in public without anyone else being aware of it.

f) If the insert is used for example to deliver medical substances, the connecting means can be manufactured longer if necessary to allow for deeper delivery, and because it is generally flexible and non-rigid in nature, it allows for far less irritation against the nasal passages than other inserts.

(g) The transparent non-rigid connecting means of our insert is harder to see and will decompose more quickly if discarded in the environment than the rigid connecting means of the past.

(h) The combination of the above advantages makes for an insert that is very user friendly and even bordering on “fun” to use, which is becoming increasingly clear at this time as a primary consideration for nasal delivery devices.

Conclusion, Ramifications, Scope

Accordingly, one will see that the intranasal insert of this invention can be used by professionals or the public alike as conveniently as the handy wipe or disposable glove. It can be left in the nose for ten minutes or ten hours, depending on the use. Moreover, unlike designs of the past, the insert genuinely provides for comfort to the user through the use of a non-rigid connecting means. Further, the insert has the additional advantages in that

upon installing, it does not irritate the nasal septum since it incorporates a flexible non-rigid connecting means;

once installed, the wearer does not experience the annoying feeling that an object is “attached” or “hanging” onto the nose;

after being installed, the insert bodies can be adjusted while inside the nose. This can be done by pulling on the connecting means and pressing with the fingers on the outside of the nose. This important ability prevents having to remove an already installed insert from the nose, thus being a more convenient and sanitary insert;

it provides for easier removal from the nose because the connecting means is non-rigid and does not clamp onto the septum nor firmly position the insert bodies into particular positions inside the nose;

it provides the user with the remarkable ability to wear the insert in front of others without being visually detectable;

it provides a connecting means that is generally less irritating to the nasal passages than inserts of the past;

it provides for a connecting means that can be far less visible and will decompose more quickly when the insert is discarded into the environment than those of inserts invented in the past.

Although the description above contains many specificities, these should not be construed as limiting the scope of the invention but as merely providing illustrations of some of the presently preferred embodiments of this invention. For example, the insert bodies can come in a vast assortment of shapes, textures, and materials, etc.; they can contain other structures, be used to absorb or gather bodily fluids, incorporate electronics including nano-technology, etc.; the connecting means can be varied by using less transparent materials, making its length adjustable as mentioned in our above patent, changing its cross-sectional shape, creating different pull members, etc.

Thus the scope of the invention should be determined by the appended claims and their legal equivalents, rather than by the examples given.

We claim:

1. An intranasal insert comprising:
a. two insert bodies which are placed inside a nose, one insert body through each nostril; and
b. in combination with at least one connecting means of a predetermined length, wherein said connecting means forms two ends, whereby each of said ends is generally connected with one of said insert bodies,
c. whereby said connecting means provides for freedom of movement and adjustability of said insert bodies inside said nose, safely prevents said insert bodies from being inhaled into said nose, and further provides a means for removing said insert bodies from said nose by forcefully exhaling airflow out of said nose or by grasping said connecting means with human fingers and pulling away said insert bodies from said nose.

2. The insert of claim 1 wherein said connecting means is made of transparent material.

3. The insert of claim 1 wherein said connecting means is composed of at least one transparent non-rigid elongated fiber such as nylon monofilament line.

4. The insert of claim 3 wherein said insert bodies may be treated with a liquid, solid, vapor, or any combination thereof, for the purpose of interacting with the human body through the respiratory system.

5. The insert of claim 1 wherein said connecting means is composed of at least one non-transparent non-rigid elongated fiber.

6. The insert of claim 5 wherein said insert bodies may be treated with a liquid, solid, vapor, or any combination thereof, for the purpose of interacting with the human body through the respiratory system.

7. The insert of claim 1 wherein said insert bodies may be enlarged to fill a section of airway through said nose.

8. The insert of claim 1 wherein said connecting means is made of translucent material.

9. An intranasal insert comprising:
a. two insert bodies of a predetermined size to be placed inside a human nose, one insert body through each nostril, and
b. at least one predetermined length of material composed of non-rigid elongated fiber, having two ends, wherein each of said ends is separately connected with one of said insert bodies.

10. The insert of claim 9 wherein said material is transparent.

11. The insert of claim 10 wherein said material is composed of transparent non-rigid elongated fiber such as nylon monofilament.

12. The insert of claim 10 wherein said insert bodies may be treated with any substance of a predetermined composition intended to influence the human body through the respiratory system.

13. The insert of claim 10 wherein said insert bodies may contain a core.

14. The insert of claim 10 wherein said insert bodies may be of enlarged size to fill a section of airway in said nose.

15. The insert of claim 9 wherein said material is composed of a plurality of non-transparent non-rigid elongated fibers.

16. The insert of claim 15 wherein said insert bodies may contain any substance of a predetermined composition intended to influence the human body through the respiratory system.

17. The insert of claim 9 wherein said material is translucent.

18. The insert of claim 9 wherein said material is extendable.

19. A method of discreet intranasal insert use which comprises the steps of:
a. providing an intranasal insert of the type comprising two insert bodies, whereby said insert bodies are connected together by a transparent connecting means,
b. inserting both of said insert bodies within a human nose, one through each nostril,
c. leaving in place said insert bodies for a predetermined time period, depending on the use and purpose of said intranasal insert,
d. removing said insert bodies by grasping said connecting means and firmly exhaling airflow out of said nose into a clean tissue for disposal, whereby said intranasal insert is virtually undetectable by persons in close proximity to the user while in use or while being removed and disposed of in said tissue, said tissue further provides a sanitary means for disposing of used said insert bodies from said nose by preventing direct contact with the fingers of said user.

20. The method of claim 19 wherein said connecting means is non-rigid.
